(19) United States
(12) Patent Application Publication
(10) Pub. No.: US 2003/0018271 A1
(43) Pub. Date: Jan. 23, 2003

(54) Simplified and lightweight system for enhanced visualization of subcutaneous hemoglobin-containing structures

(76) Inventor: Allan Wayne Kimble, Jacksonville, FL (US)

Correspondence Address: ALLAN WAYNE KIMBLE, 5085 BRADFORD ROAD, JACKSONVILLE, FL, 32217, US

(21) Appl. No.: 10/183,622

(22) Filed: Jun. 28, 2002

Related U.S. Application Data
(60) Provisional application No. 60/302,284, filed on Jul. 2, 2001.

(51) Int. Cl. A61B 6/00

(52) U.S. Cl. 600/473

(57) ABSTRACT

A simplified, lightweight and inexpensive system and method for enhancing visualization of veins or other subcutaneous natural or foreign structures of the body containing hemoglobin is provided. This system will facilitate intravenous insertion or extraction of fluids, medication or other treatments in hospital or emergency settings.

This invention claims the benefit of earlier filed U.S. Provisional Application No. 60/302,284 filed Jul. 2, 2001, incorporated herein by reference.

### FIELD OF THE INVENTION

The invention described herein relates generally to medical devices and procedures, and more particularly to a simplified system and method for enhancing visualization of veins and other subcutaneous hemoglobin-containing features of the body. Such visualization will aid in fluid insertion into or extraction from the body. Subcutaneous blood or hemoglobin containing areas can provide useful information for diagnosis of the medical condition of a patient or administration of medical treatment to a patient.

### BACKGROUND ART

The earliest efforts to use infrared to locate veins and blood were led by Eastman Kodak Company™ and used photography to accomplish venous visualization. “Medical Infrared Photography,” published by them is incorporated into this patent by reference. (Eastman Kodak Co; ISBN: 0879850256; 3rd edition, June 1973.) Other prior art devices and procedures for enhancing visualization of veins, arteries and other blood or hemoglobin containing subcutaneous structures of the body have included the following techniques: applying tourniquets, flashlights, direct application of liquid crystalline materials, dual fiber optic sources, ultrasonic imaging and nuclear magnetic resonance imaging. The tourniquet method is the traditional approach in which the venous return is restricted to cause the major superficial venous vessels to engorge with blood for enhancing their visibility This is the standard approach used in all medical facilities. However, this technique is compromised in conditions of poor ambient illumination, and inpatients with low blood pressure, racial pigmentation, skin bums, etc. Flashlights are limited to transilluminating very thin sections of tissue. The liquid crystal technique is based on the thermal sensitivity of liquid crystal materials. By applying a thin liquid crystal film over the vein, it is possible to map out the venous structure from the surrounding tissue based on relative temperature differences. The dual fiber-optic source is a method by which both sides of the venous structure are simultaneously illuminated with visible light to eliminate shadows and to provide enhanced visualization. Ultrasonic images can be taken of vascular and surrounding tissue. This technique is based on the reflection of ultrasonic waves due to the impedance mismatch at the various tissue interfaces found within the body. Lastly, the nuclear magnetic resonance (NMR) imaging technique relies on the magnetic relaxation times of various chemical species specific to blood within the body.

In U.S. Pat. No. 4,619,249 Landry describes a method of illuminating veins to aid in their visualization with the unaided eye. The awareness of difficulties in use of that invention with persons with dark racial skin pigmentation are evident in that the mention of the use of more intense lighting to achieve the desirable result.

In U.S. Pat. No. 5,217,456 Narciso describes a system limited to intra-vascular imaging and is a medically invasive technique. Although the method described demonstrates the internal structures of the vascular network, it is limited to those structures large enough to admit a catheter and in doing so, carries an inherent risk not found in the present invention that is wholly non-invasive and non-contact during the typical manner of use.

The invention by Shimizu described in U.S. Pat. No. 5,337,749 utilized nuclear magnetic resonance technology. The typical NMR system comprises a large, massive magnet and sensor system to produce tomographic slices that are then deconvoluted into an image. A typical NMR system is prohibitively expensive so as to preclude its widespread use except in special imaging centers or major hospital or university settings.

The invention by Esparza described in U.S. Pat. No. 5,519,208 utilizes filtered and focused light as well as a second filtering mechanism to form the images of the body parts being examined. Additional, the invention by Esparza uses visible wavelengths for imaging. The invention by Corso described in U.S. Pat. No. 5,876,346 uses means other than optical to locate and characterize arteries. The method is limited to arterial location rather than visualization.

The invention described by Groner, et al., in U.S. Pat. No. 6,104,939 like that of Esparza utilizes visible wavelengths of light and specifically cites 550 nm and 650 nm as the preferred wavelengths. In addition to the use of visible radiation, the system of Groner, et al. also requires a polarizer to improve contrast.

Similarly, the invention of Jacques described in U.S. Pat. No. 6,177,984 utilizes polarized light that is scattered from living tissue to achieve selective vascular imaging. Additionally, an optical retarder is used in conjunction with the other optical elements to produce an image that can be further enhanced using computerized means. A computer system is necessary for the system to produce easily interpretable vascular images.

In the invention by Svetliza, described in U.S. Pat. No. 6,178,340 a three-dimensional image is formed using at least a pair of sensors and also includes a computer to process the data from the sensors. In the invention of Svetliza, a stereo image utilizing red-green imaging and complementary glasses are used to recreate the proper parallax of natural vision and its associated depth perception.

The invention of Crane, et al. described in U.S. Pat. No. 6,230,046 represents a complicated and technically limited approach to spectral imaging of the vascular system. For example, Crane cites the use of an image intensifier tube, and later describes night-vision goggles. Although image intensifiers can detect and display images of weakly luminous sources, such as transilluminated tissues and body parts, there is nothing inherent in their response that limits their response to vascular tissues other than the optical density in the visible portion of the spectrum of blood, bone and other optically dense subcutaneous structures. Crane next mentions a photomultiplier tube and photodiode. Each of these devices provides only a single intensity measurement, regardless of the optical components placed between them and the object being examined. To form an image, the detector must be scanned or the body part being examined moved through the field of view of the sensor and the image assembled as a mosaic. The invention of Crane et al. mentions the use of a charge-coupled device, or CCD to perform the detection function of the system. However, Crane describes the CCD as a low-light-level detector. Although a specific class of CCD's exist that can be described as low-light-level detectors, these usually require additional hardware or processing such as thermoelectric cooling, back-thinning of the detector or back-illumination of the sensor. Crane et al., specifically cites a bandpass filter to select the wavelengths of interest. This type of filter is costly to implement and sensitive to angular placement in the optical system. It is relatively sensitive to changes in temperature that can cause the bandpass to change, thereby jeopardizing the optimal contrast of the system. Lastly, the figure in the Crane invention illustrating the head-mounted night-vision goggle system is complex, heavy, and cumbersome to use.

There still exists a need for a simple, lightweight hemoglobin visualization system to be present in emergency and regular health care facilities.

### SUMMARY OF THE INVENTION

While sorting through several wavelength and detector responses during preparation for a presentation to an astronomy group on the topic of lunar meteor impact imaging, it was unexpectedly noted that when an experimentally assembled near-infrared (NIR) illumination and detection system was pointed at the exposed skin of a human, the venous system was clearly shown. This result was a complete surprise and resulted in tests of the device on several people present in my garage. The results were clear and definitive. Blood rich tissue clearly showed up well on the image monitor. The system giving these results was comprised of the following:

Tri-Tronics™ HSLS-12 Super High Intensity Light Source

Topward™ 3302D DC Power Supply

Ikegami™ Model PM-960 Monochrome Monitor

Computar™ 8 mm 1:1.2 ⅓-inch CS lens

Generic 12VDC power supply for the detector

Kodak™ Wratten Infrared Filter No. 87C

Watec™ LCL-903HS Charge Coupled Device (video camera)

Although a light source was used in the initial trials of the device, it was quickly noted that it was not necessary as long as sufficient ambient light having a near-infrared spectral component was present. Sunlight, incandescent light and certain fluorescent lights were found to be quite effective as sources of illumination, even at normal ambient levels such as that found in homes, offices and medical facilities. Later experimental efforts clearly showed the ability of this system to indicate the presence of infections and bruises. It was also realized that the system described above could be miniaturized and produced very inexpensively. This seemed to be a potential candidate for commercial use.

The basic invention is therefore:

A blood detection system comprising a band or long-pass filter for near infrared radiation located between the viewed subject and the detector, a camera-like detecting system sensitive to said passed infrared radiation and other selected wavelengths, and an imaging system for displaying the passed wavelength patterns in human viewable format.

It is therefore a principal object of the invention to provide a simplified and lightweight system and method for the non-invasive visualization or identification of subcutaneous hemoglobin-containing features of the body.

It is another object of the invention to provide a system and method for detecting and mapping the veins other subcutaneous hemoglobin-containing structures in human or animal subjects.

It is another object of the invention to provide a system and method for visualization and identification of veins and other subcutaneous hemoglobin-containing structures under adverse lighting conditions.

It is another object of the invention to provide a non-invasive means to visualize human body internal tissue containing hemoglobin for purposes of diagnosis, and/or administration of medical treatment, or surgery. It is a further object of the invention to provide system and method for intravenous insertion or extraction of fluids under adverse lighting conditions or in lighting conditions normally found in hospital environments (such as wards, emergency, operating, and recovery rooms) for magnified or non-magnified visualization.

It is a further object of the invention to provide a system and method for insertion or extraction of fluids in an emergency situation or to patients/victims who are difficult to catheterize or phlebotomize.

It is a further object of the invention to provide system and method for insertion or extraction of fluids in a hospital environment (such as removal of lymphatic fluid/blood from internal injury).

It is a further object of the invention to provide system and method for insertion or extraction of fluids in a mobile environment (such as an ambulance, aircraft or other medical emergency conveyance).

It is another object of the invention to provide system and method for visualization and identification of foreign bodies and medical appliances in the body that may block the optical path of hemoglobin-containing subcutaneous structures.

It is another object of the invention to provide system and method for visualization and identification of veins and other subcutaneous hemoglobin-containing structures in classes of patients in which the vascular system is difficult to visualize (such as pediatric, geriatric, racially-pigmented, obese, burn, etc).

It is yet another object of the invention to provide system and method for visualization and identification of veins and other subcutaneous hemoglobin-containing structures in animal subjects.

It is yet another object of the invention to provide system and method for visualization and identification of veins resulting from a process known as neovascularization related to cancerous processes within the body.

These and other objects of the invention will be more fully appreciated by one skilled in the applicable art as a detailed description of representative embodiments thereof proceeds

### BRIEF DESCRIPTION OF THE DRAWINGS

FIG. 1 illustrates a representative embodiment of the invention for the visualization of subcutaneous blood containing structures of the human body, such as the shoulder and forearm shown.

FIG. 2 is a view of three possible views the present invention is capable of displaying.

FIG. 2A illustrates the display of normal venous structure of the forearm.

FIG. 2B illustrates the display of trauma resulting in leakage of blood into the surrounding tissues, e.g. bruising.

FIG. 2G illustrates the display of neovascularization resulting from certain types of cancers and malignant neoplasms.

FIG. 3 is a diagram of the lightweight head mounted viewing system that allows the wearer to observe subcutaneous hemoglobin-containing structures while leaving the hands free to conduct medical procedures such as surgery, administration or removal of fluids.

FIG. 4 illustrates a portable hand-held or otherwise mounted version of the device.

### DETAILED DESCRIPTION OF THE INVENTION

In accordance with a governing principle of the invention, subcutaneous blood containing structures, such as vein or artery structures in soft tissue of the body or cysts, cancers, tumors, or other abnormal structures may be visualized by utilizing the property of blood to absorb specific spectral wavelengths. Illuminating or transilluminating the corresponding body portion with light of appropriate spectral composition is required, but such wavelengths are often present under normal lighting conditions. If a light source is needed, it does not have to be intense or physically threatening and may be in non-limiting examples from an LED, laser, chemiluminescent, incandescent or fluorescent source (IES Lighting Handbook, J. E. Kaufman, Ed., Illuminating Engineering Society of North America, New York, 1984). Viewing will require a camera-like system having: 1) a selected band or longpass filter, 2) a spectrally selective array-type imaging device responsive to the spectrum chosen, and 3) an image forming system such as those common to computers or computer games.

It is projected that the whole system will weigh less than 2 Kg, probably less than 0.5 Kg. If a full size TV-type monitor is used for image display, then the weight may be greater.

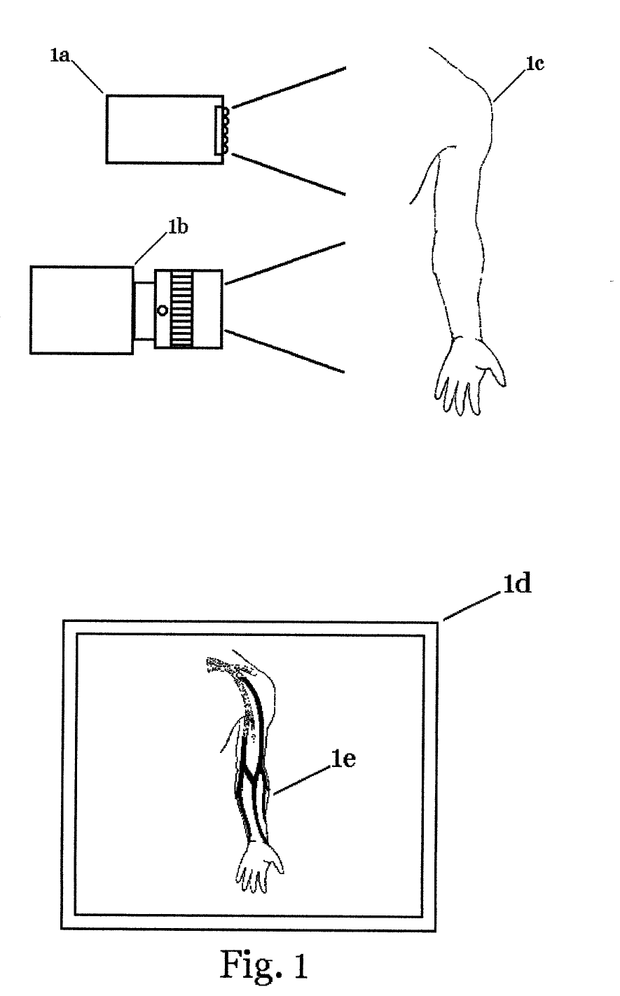

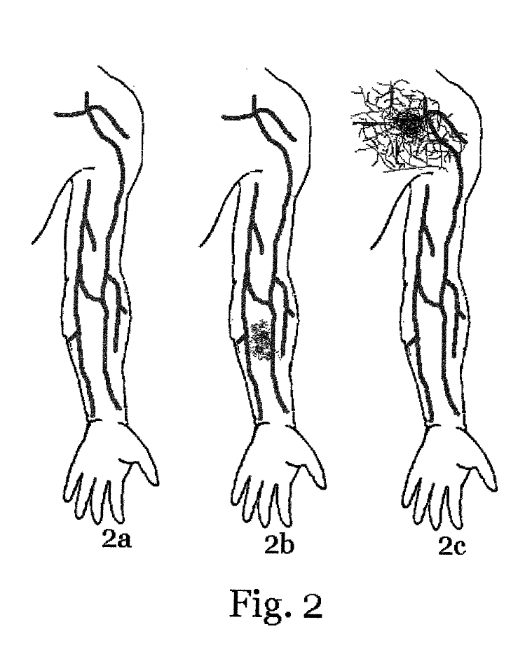

FIG. 1 illustrates a representative system 1b of the invention usable by an observer in viewing subcutaneous structures of the human body 1c (represented by the hand, forearm and shoulder shown) utilizing a spectrally selective array-type detection means represented by a modified commercial ambient-light level charge coupled device (CCD) camera. The system may therefore include light source 1a of selected wavelength(s) in the near infra-red (NIR), depending on the type of detector array 1b used and on the particular subcutaneous structure of interest. Body portion 1c containing the structure to be visualized may be illuminated directly (in a reflection mode) or where the thickness (and consequent degree of absorption and/or scatter) of the hemoglobin-containing tissue of body portion 1c permits, by transillumination through body portion 1c. In the practice of the method of the invention, source 1a may be placed near body portion 1c to illuminate the surface thereof in the reflection mode of operation. Alternatively light source 1a is placed near or against the surface of body portion 1c opposite the surface to be viewed so that light from source 1a is transmitted through body portion 1c. As shown in FIG. 1 the light source is placed outside of the hand, arm and shoulder and the viewer is placed facing the said body parts to view the veins 1e of the same. The orientation or illumination direction is chosen depending on the subcutaneous structure that is to be viewed. Because of the differences in absorption characteristics among venous blood, arterial blood, and any abnormal structures as compared to the skin, bone and surrounding muscle and fatty tissue, the visualization of the location and arrangement of the veins 1e, or other hemoglobin-containing structures may be visualized using CCD of appropriate spectral sensitivity. FIG. 5 details the various components that comprise the preferred embodiment of the invention. A source of electrical power 5a is connected to the circuitry of the system via a pair of wires 5b. The various electronic components 5c necessary for conversion of the detector signal are mounted on a circuit board 5e. A mechanical means Se for attachment of a lens, mirror or combination thereof 5g, is also attached to the circuit board 5d. A typical means of attachment is through the use of threaded mating surfaces (5f and 5I) The optical focusing mechanism may also contain an iris diaphragm 5h to control the quality of the image formed on the detector array. An optical filter 5j of the interference structure type (such as fabricated using rugate or stack technology, yielding filter types such as bandpass (cavity, Fabry-Perot, induced transmission) low pass, high pass, band stop, or tunable filters that may be found by reference to W. E. Johnson et al, “Introduction to Rugate Filter Technology, Inhomogeneous and Quasi-Inhomogeneous Optical Coatings,” Proc. SPIE 2046, 88-108 (1993); or to H. A. Macleod, Thin Film Optical Filters, 2nd Ed, Macmillan, N.Y. (1986)), or an absorbing structure (which may be found by reference to Schott Glass Technologies, Inc., Optical Glass Filters, Dureya, Pa. (1986) and Infrared Dyes, M. Matsuoka, Ed., Plenum Press, New York (1990)), or a combination of the two, may be used to select the spectral range of viewing into transmission band(s) to allow use of system with natural or artificial light sources, to differentiate venous from arterial blood or to exclude noise or other radiation not contributing to the desired image. It is noted that filtering competing light sources in the passband(s) of interest improves the contrast ratio or signal-to-noise ratio for the system. The absorbing structure can be the substrate (such as polymers, amorphous or crystalline ceramics, semiconductors or composites) and/or optical coating while the interference structure is typically the coating. The specific filter for accomplishing a particular spectral sensitivity may be selected by one skilled in the applicable art guided by these teachings, the same not considered limiting of the invention herein. Ambient light may be excluded from the spectral range of interest by performing the method of the invention in an environment suitably shielded by filter means represented in FIG. 5 by filter 5j.

Image Creation

In the contemplation of the invention, the image of the scene can be visualized utilizing various spectrally selective array-type light detection means. In a first such mean, viz., a stating system, a lens is placed in front of a detector array such as a CCD. The position of each element of the scene is registered with the position of the image on the output display such as a cathode ray tube or liquid crystal display device.

Charge-Coupled Devices

The image sensing capability of the CCD is based on the absorption of incident radiation in the silicon that generates a linearly proportional number of free electrons in the specific area of illumination. The CCD array is composed of a repetitive pattern of small photosensing sites, each generating a charge packet in direct response to the incident radiation. By creating an image of the external scene on the array, the charge packet distribution in the array will reproduce the light distribution in the scene. At regular intervals, the charge packets along one column of the array are simultaneously transferred by charge coupling to a parallel CCD analog transport shift register. The photosites are then returned to a new iteration of image collection. While the photosites are collecting a new image, the CCD analog transport register is rapidly clocked to deliver the picture information in serial format to the device output circuitry. The output circuit delivers a sequence of electrical signals in which the amplitude is proportional to the amount of charge generated at each photosite. By mapping the signal back to the individual photosites, it is possible to recreate the scene image. The CCD does not need to be of the low light level design, as the sensitivity of standard CCD's is sufficient to achieve acceptable imaging of hemoglobin-containing subcutaneous structures when appropriate lighting and filtering is available. The use of image intensifiers, such as those used in night vision goggles (NVG's) is not preferred since the NVG is intrinsically electronically noisy, and though suitable for gross imaging needs in military applications, renders high-resolution imaging of vascular structures difficult. Additionally, the size, weight and generally unwieldy character of NVG's make them impractical for routine medical procedures. Other types of low-light level CCD's also require additional circuitry and often require cooling to reduce electrical noise that can otherwise degrade the image quality. These elements are capable of producing images of hemoglobin-containing subcutaneous structures, though not necessary for the preferred embodiment of this invention.

Charge Injection Devices

Charge injection devices (CID's) are similar in performance to CCD's but do not suffer from some inherent drawbacks (e.g., blooming) of CCD's and offer advantages (e.g., non-destructive readout) not possible with CCD's. The spectral sensitivity of the CID makes it useful array detector for the present invention. The pixels of the CID are front-illuminated since there is only a small amount of gating structure on the top surface to obstruct incoming photons. A thin metal strip is put on top of the row polysilicon to reduce the readout noise. The drawback to this opaque structure is a small amount of obstruction of the incoming light. Each pixel consists of a pair of orthogonal polysilicon electrodes that create two MOS capacitors in n-doped silicon for storage and sensing of photo-generated charge. These electrodes also connect the rest of the pixels on the column or row to the scanners on the periphery. Integration of photo-generated holes occurs in the positively biased epitaxial region. Since the substrate is grounded, a reverse biased p-n junction is created inside of every pixel. This provides excellent anti-blooming protection when overexposed since excess holes outside the well are swept through the p-n junction to the substrate. Negative or slightly positive voltages on the column and row electrodes create depletion wells for storage of holes. In preamp per row (PPR) devices, columns are biased more negatively and holes collect under this electrode called the “collection pad”. The same FET amplifies all pixels along a particular row and hence the PPR architecture requires slight calibration of the 512 row FET's to minimize nonuniformities between them. Further reduction of read noise can be achieved with preamp per pixel (PPP) structures. There are two types of readout techniques. During readout, a “zero level” is captured on the sense pad along the row by allowing the pad to float and digitizing. Driving the column high performs the sole charge-transfer from the collection-well to the sense-well and all stored charge moves to the sense-well. The amount of collected charge sensed on the row-electrode modulates the drain-source current of the output FET amplifier. In nondestructive readout (NDRO) the low potential on the collection pad is reestablished and accumulation continues. In destructive readout (DRO), the pixel is injected. This information is available from CIDTEC, a manufacturer of CID array detectors and is incorporated herein by reference.

Photodiode Arrays

Photodiode arrays are generally useful for low light detection and operate in the range of about 200 to 1105 nanometers (nm). The diode junction acts as a photodetector. An electron hole pair can be created by an incident photon provided that the photon energy is greater than the semiconductor band gap energy. This can occur in any of the semiconductor layers. Once carriers are created, a current will flow until they are collected or recombined. Two basic types of photodiodes are typically used: silicon PIN photodiodes and the silicon avalanche photodiodes (APD). At low frequencies and at low but not ultralow signal levels, a PIN photodiode is preferred. At lower light levels, avalanche photodiodes are preferred. A high reverse bias voltage leads to a high field in the p-n junction region. Photogenerated or thermally generated carriers that reach this region are accelerated to energies at which collisional ionization occurs resulting in a multiplication of carriers thus resulting in internal gain. APDs can have quantum efficiencies in excess of 90% and noise equivalent powers less than 10.sup.−15 W/Hz.sup.−½. The use of an array rather than a single or discrete photodiode precludes the need to scan or otherwise move the detector or object being observed in order to generate an image.

Light Source

Selection of the light source (FIGS. 1, 1a) for illuminating (in the reflection mode) or transilluminating the body portion (FIGS. 1, 1a) of interest may also be made by the skilled artisan practicing the invention in consideration of the intended use of the system in visualizing a particular hemoglobin-containing subcutaneous structure, such as for facilitating the location of a vein for insertion of an intravenous needle for blood transfusion, administration of an injection or other medication or determination of the location and extent of neovascularization. Observations made in demonstration of the invention in the reflection mode for an NIR light source showed that sufficient contrast to show veins in the hand, forearm, shoulder, neck and legs could be achieved over the 0.65 to 0.95 .mu.m band using a CCD fitted with a long-pass glass or polymer filter; above about 1.0 .mu.m the CCD response falls off. Similar demonstration experiments proved the utility of the invention to allow visualization of an abnormal vein growth in a human female leg. An NIR source is preferred because of the hemoglobin-specific radiation absorption associated with an NIR source than with sources of shorter wavelength, NIR exhibits better transmission characteristics through body tissue than visible or UV, CCD's and ClD's operate efficiently in the NIR below approximately 1.0 .mu.m. Human skin readily transmits NIR and the underlying or subcutaneous hemoglobin-containing structures absorb NIR when they contain deoxygenated hemoglobin.

The contrast ratio or signal-to-noise-ratio (SNR) drives the spectral performance of both source and filter. For example, using a narrow band light source, such as a laser emitting diode, and a filter having passband(s) which are very narrow (a few nanometers FWHM) and highly transmitting (>80%) will yield a good SNR. Filters having high attenuation (about 10.sup.−4 to 10.sup.−5) outside of the passband(s) will further improve the SNR. Preferably, the method of contrast enhancement is the removal of unwanted optical radiation below 0.65 .mu.m. and allowing only wavelengths that correspond to the optical absorption of hemoglobin and within the sensitive spectral range of the array-type detector. Increasing the illuminance of the background lighting; such as found in a windowless but, highly lit operating room at a hospital, decreases the SNR for systems that allow optical radiation in the visible portion of the spectrum to reach the detector. However, lighting covers that transmit visible wavelengths (>80%) but, highly attenuate NIR wavelengths (about 10.sup.−4 to 10.sup.−5) negate this decrease in the SNR. These lighting covers can be either glass, such as Schott BG 39 or BG 40, or a polymer or plastic, e.g., polymethylmethacrylate, impregnated with materials such as nickel dithiolene complexes. In general, incandescent rather than fluorescent lighting is preferred. The entire teachings of all references cited herein are incorporated herein by reference.

### INDUSTRIAL APPLICABILITY

The invention therefore provides system and method for enhanced, non-invasive or invasive surgical procedures (e.g., angiography) visualization or identification of subcutaneous hemoglobin-containing structures of the body. In the practice of the invention, the detection, identification and mapping of veins and other subcutaneous hemoglobin-containing structures in human or animal subjects may be performed under adverse lighting conditions associated with the emergency administration of medical treatment or in lighting conditions that could be found in hospital environments, for purposes of diagnosis, administration of medical treatment, and surgery, or for visualization and identification of foreign bodies and medical appliances in the body that either contain hemoglobin, or block or partially block the view of hemoglobin-containing structures.

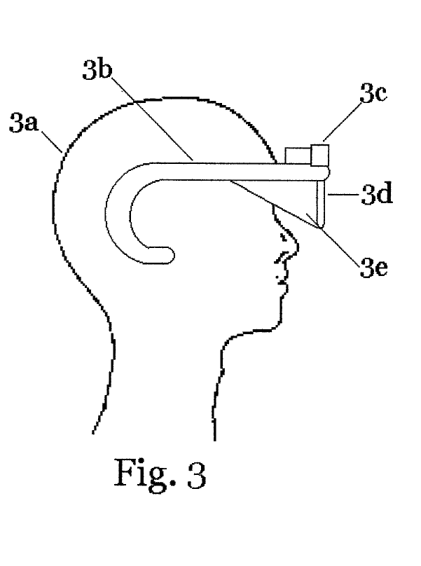

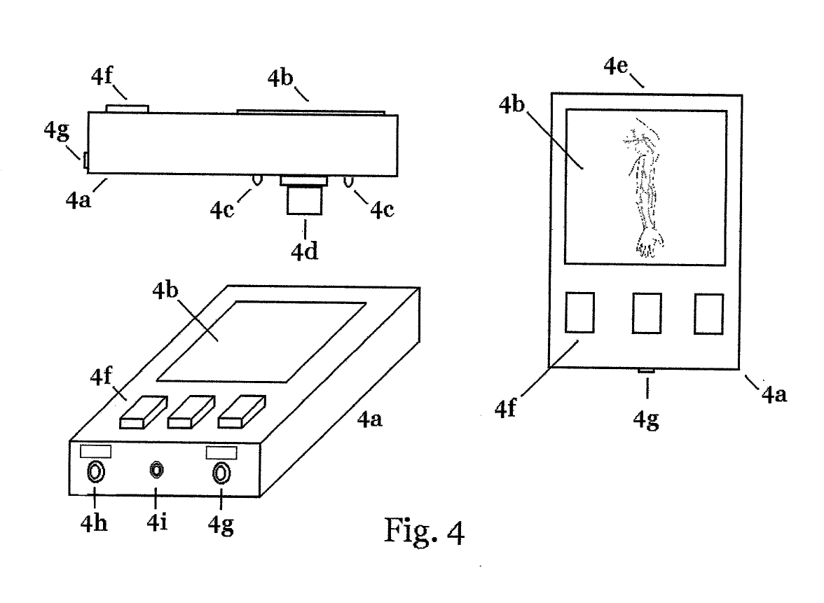

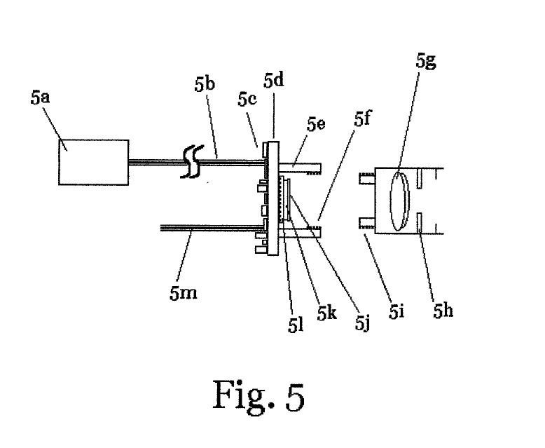

The preferred embodiment of this invention is shown in FIG. 3 that illustrates the lightweight head-mounted version of the system. This embodiment is shown being worn by an observer 3a wherein the system is held in place upon the head of the observer through the use of earpieces 3b such as are commonly found on spectacles. The spectrally selective array-type detector 3c is integrated into the system and is lightweight, preferably less than two kilograms, more preferably less than one kilogram. A display device 3d consisting of liquid crystal displays (LCD's) is positioned such that the observer may comfortably view the image generated by the detector array 3c. A single detector array 3c or a pair of detector arrays may be used to generate either a monovision or stereovision display, respectively by sending their signals to the appropriate display device 3d. The display unit can be either opaque, thereby restricting the observer to only the electronic images generated by the detector(s) or semi-transparent allowing the observer to see the enhanced visualization of subcutaneous structures overlaid upon the view of the body part(s) being examined. The system may also use a single detector and display device for one eye, leaving the unenhanced view through the device of the body part being examined for the remaining eye. Optical shielding 3e is provided to reduce peripheral light from reaching the observer's view of the display(s). The principle advantages of this invention over the prior art are the ease of use, lightweight and hands-free operation. The latter advantage allows the observer to maintain constant natural view of the body part being examined while enabling the observer to use their hands to perform any procedures that might be required. The advantage of being lightweight allows the observer to use the device for extended periods of time without undue stress and strain on the head and neck that might be expected from heavier devices described in the prior art. Finally, the preferred embodiment provides an appearance not unlike sunglasses or safety glasses that puts the recipient of the examination at ease during the examination, as opposed to military-style NVG's that were not designed with the intention of patient examination. Another embodiment of the device is shown in FIG. 4. In this embodiment, the system is designed to be hand-held for examination purposes. The system is small and self-contained and allows an observer to manually scan various body parts to determine the location and characteristics of subcutaneous hemoglobin-containing structures. The housing 4a contains the power supply, e.g., a battery or similar electrical energy storage device, the display device 4b that can be any suitable device, e.g., an LCD, plasma display or CRT, supplementary lighting 4c with at least a portion of the radiated energy being spectrally located in the NIR region of the spectrum, i.e., 650 nm-1100nm, a focusing mechanism 4d to form an image on the detector array. The system is normally held with the top of the device 4e positioned as shown in FIG. 4 and the focusing mechanism 4d aimed at the body part to be examined. The system controls 4f allow the observer to power the system on and off, freeze the image by storing it into a temporary buffer and to add supplementary lighting if needed. The system can be operated using a remote power supply plugged into port 4g that can be also used for recharging the internal batteries of the device. Should the image be required to be sent to a computer or printer for long-term storage or analysis, a video port 4h compatible with any of the various video standards is also supplied. A standard camera mounting (¼-20 thread) 4i, is supplied for mounting of the device onto a tripod for extended viewing times and to free the hands of the observer for the performance of various tasks that may be required.

It is understood that modifications to the invention may be made as might occur to one having skill in the field of the invention within the scope of the appended claims. All embodiments contemplated hereunder that achieve the objects of the invention have therefore not been shown in complete detail. Other embodiments may be developed without departing from the spirit of the invention or from the scope of the appended claims.

What is claimed is:

1. A system for enhanced viewing of hemoglobin containing structures within human or animal tissue comprising:
a) a filter to pass wavelengths between 650 and 1100 nm;
b) a lens assembly designed to focus an image of said human or animal tissue upon an array-type detector responsive to wavelengths passed by said filter, said detector designed to deliver its output to a display device;
c) a display device for converting said detector output into an image useful for medical treatment or study;
whereby rapid and accurate location of blood containing structures is facilitated.

2. The system of claim 1 where the weight of all items excluding the display device is less than 2 kilograms.

3. The system of claim 1 where the weight of all items is less than 4 kilograms.

4. The system of claim 1 where the source of light is ambient, natural or artificial, containing at least some energy in the near-infrared portion of the electromagnetic spectrum between 650 nm and 100 nm;

5. The system of claim 1 where the focusing means comprises at least one device for formation of an image on an array detector consisting of a lens, mirror or combination of lenses and mirrors;

6. The system of claim 1 where the spectrally selective array detector is a device selected from the group consisting of a charge coupled device, a charge injection device, a microbolometer array device, a platinum silicide detector array, an indium antimonide detector array, a copper germanium detector array, deuterated triglycine sulfate detector array, mercury cadmium telluride detector array, a densely packed phototransistor array, a densely packed silicon photodiode array, a quantum dot detector array, a nanotube detector array, an yttrium barium copper oxide detector array, a gallium arsenide detector array, an aluminum gallium arsenide detector array, a cadmium zinc telluride detector array, a photoconductor detector array, and a photovoltaic detector array.

7. The system of claim 1 where the filter is a device selected from the group consisting of long-pass filters composed of polymers, amorphous or crystalline ceramics, semiconductors or composites that pass wavelengths greater than 650 nm; or at least one spectrally selective device means includes a device selected from the group consisting of band-pass filters composed of polymers, amorphous or crystalline ceramics, semiconductors or composites that pass wavelengths greater than 650 nm but less than 1100 nm;

8. The system of claim 1 wherein said at least one spectrally selective array-type light detection means is one of a monocular arrangement providing independent viewing of two spectral visual fields of said image and a binocular arrangement providing depth perception of said image.

9. A system for enhancing the visualization of veins and other subcutaneous natural or foreign hemoglobin-containing structures in the body, comprising:
(a) a natural or artificial light source for illuminating a portion of the body in at least one of a reflection mode or a transillumination mode;
(b) at least one spectrally selective array-type light detection means for detecting light having spectral characteristics defined by the relative light absorption properties of subcutaneous hemoglobin-containing structures within said portion of the body, said light reflected from or transmitted through said portion of the body, and for generating an image of subcutaneous hemoglobin-containing structures within said portion of the body, and means for displaying said image; and
(c) an optical filter disposed between said portion of the body and at least one spectrally selective array-type light detection means for transmitting light only within preselected narrow wavelength bands.

10. The system of claim 9 wherein said natural or artificial light source includes said preselected broad wavelength bands and said at least one array-type light detection means is sensitive to said preselected broad wavelength bands.

11. The system of claim 9 wherein said source is a source selected from the group consisting of a light emitting diodes and near-infrared lasers.

12. The system of claim 9 wherein said optical filter has a long passband beginning transmission substantially on at least one wavelength selected from the group consisting of 0.65, 0.70, 0.75 and 0.81 and 0.85 microns.

13. The system of claim 9 wherein said natural or artificial source is a broadband source selected from the group selected from an incandescent source, a chemiluminescent source and a fluorescent source having sufficient energy in a spectral band greater than 0.65 micron up to about 1.1 microns.

14. The system of claim 9 wherein said at least one spectrally selective array-type light detection means is one of a monocular arrangement providing independent viewing of two spectral visual fields of said image and a binocular arrangement providing depth perception of said image.

15. A system for enhancing the visualization of veins and other hemoglobin-containing subcutaneous natural or foreign structures in the body, comprising:
(a) a natural or artificial light source for illuminating a portion of the body in at least one of a reflection mode or a transillumination mode;
(b) at least one spectrally-selective array-type light detection means for detecting light having spectral characteristics defined by the relative light absorption properties of subcutaneous hemoglobin-containing structures within said portion of the body, said light reflected from or transmitted through said portion of the body, and for generating an image of subcutaneous hemoglobin-containing structures within said portion of the body, and means for displaying said image; and (c) means for filtering ambient light at said spectrally-selective array-type detector from said at least one spectrally-selective array-type light detection means.

16. The system of claim 15 wherein said at least one spectrally selective array-type light detection means is one of a monocular arrangement providing independent viewing of two spectral visual fields of said image and a binocular arrangement providing depth perception of said image.

17. A system for enhancing the visualization of veins or other subcutaneous hemoglobin-containing structures in the body, comprising:
(a) a natural or artificial light source for illuminating a portion of the body in at least one of a reflection mode and a transillumination mode, said light source including preselected wavelength bands;
(b) at least one spectrally selective array-type light detection means for detecting light having spectral characteristics defined by the relative light absorption properties of hemoglobin-containing subcutaneous structures within said portion of the body, said light reflected from or transmitted through said portion of the body, and for generating an image of subcutaneous hemoglobin-containing structures within said portion of the body, and means for displaying said image;
(c) an optical filter disposed between said portion of the body and said at least one spectrally selective array-type light detection means for transmitting light only within said preselected wavelength bands; and
(d) wherein said optical filter has a broad passband beginning transmission substantially on at least one wavelength selected from the group consisting of 0.65, 0.70, 0.75 and 0.81 and 0.85 microns.

18. A system for enhancing the visualization of veins or other subcutaneous hemoglobin-containing structures in the body, comprising:
(a) a natural or artificial light source for illuminating a portion of the body in at least one of a reflection mode and a transillumination mode, said light source including preselected wavelength bands;
(b) at least one spectrally selective array-type light detection means for detecting light having spectral characteristics defined by the relative light absorption properties of subcutaneous hemoglobin-containing structures within said portion of the body, said light reflected from or transmitted through said portion of the body, and for generating an image of subcutaneous hemoglobin-containing structures within said portion of the body, and means for displaying said image;
(c) an optical filter disposed between said portion of the body and said at least one spectrally selective array-type level light detection means for transmitting light only within said preselected wavelength bands; and
(d) wherein said at least one spectrally selective array-type light detection means includes a device selected from the group consisting of a charge coupled device, a charge injection device, a microbolometer array device, a platinum silicide detector array, an indium antimonide detector array, a copper germanium detector array, deuterated triglycine sulfate detector array, mercury cadmium telluride detector array, a densely packed phototransistor array, a densely packed silicon photodiode array, a quantum dot detector array, a nanotube detector array, an yttrium barium copper oxide detector array, a gallium arsenide detector array, an aluminum gallium arsenide detector array, a cadmium zinc telluride detector array, a photoconductor detector array, and a photovoltaic detector array.

19. The system of claim 18 wherein said spectrally selective array-type detector is a charge coupled device.

20. The system of claim 18 wherein said at least one spectrally selective array-type light detection means is one of a monocular arrangement providing independent viewing of two spectral visual fields of said image and a binocular arrangement providing depth perception of said image.

21. A method for enhancing the visualization of veins, or other subcutaneous hemoglobin-containing structures of the body, comprising the steps of:
(a) providing a natural or artificial source of light in a wavelength range of about 0.65 to 1.1 microns;
(b) illuminating a selected portion of the body in at least one of a reflection mode and a transillumination mode with light from said source;
(c) detecting light reflected from or transmitted through said portion of the body, said light having spectral characteristics defined by the relative light absorption properties of subcutaneous hemoglobin-containing structures within said portion of the body;
(d) generating an image of subcutaneous hemoglobin-containing structures within said selected portion of the body;
(e) displaying said image; and
(f) wherein the step of detecting light is performed using at least one spectrally-selective array-type light detection means sensitive to selected wavelength bands within said wavelength range.

22. The method of claim 21 wherein said at least one spectrally-selective array-type light detection means includes a device selected from the group consisting of a charge coupled device, a charge injection device, a microbolometer array device, a platinum silicide detector array, an indium antimonide detector array, a copper germanium detector array, deuterated triglycine sulfate detector array, mercury cadmium telluride detector array, a densely packed phototransistor array, a densely packed silicon photodiode array, a quantum dot detector array, a nanotube detector array, an yttrium barium copper oxide detector array, a gallium arsenide detector array, an aluminum gallium arsenide detector array, a cadmium zinc telluride detector array, a photoconductor detector array, and a photovoltaic detector array

23. A method for enhancing the visualization of veins or other subcutaneous hemoglobin-containing structures of the body, comprising the steps of:
(a) providing a natural or artificial source of light in a wavelength range of about 0.65 to 1.1 microns;
(b) illuminating a selected portion of the body in at least one of a reflection mode and a transillumination mode with light from said source;
(c) generating an image of subcutaneous structures within said selected portion of the body;
(d) selectively filtering light from said selected portion of the body;
(e) displaying said image; and
(f) wherein the steps of generating an image and displaying said image are performed using spectrally-selective array-type light detection means for detecting light having spectral characteristics defined by the relative light absorption properties of subcutaneous hemoglobin-containing structures within said portion of the body, said low light levels reflected from or transmitted through said portion of the body.

24. The method of claim 23 wherein the step of selectively filtering light reflected from or transmitted through said selected portion of the body is performed using a filter having a broad passband beginning transmission substantially on at least one wavelength selected from the group consisting of about 0.65, 0.70, 0.75 and 0.81 and 0.85 microns.
